(19) United States
(12) Patent Application Publication
(10) Pub. No.: US 2002/0164368 A1
(43) Pub. Date: Nov. 7, 2002

(54) Male contraceptive method and composition

(76) Inventor: Ronald Zimmerman, Greenwood, IN (US)

Correspondence Address: Intellectual Property Group, Bose McKinney & Evans LLP, 2700 First Indiana Plaza, 135 North Pennsylvania Street, Indianapolis, IN, 46204, US

(21) Appl. No.: 10/022,671

(22) Filed: Dec. 17, 2001

Related U.S. Application Data
(60) Provisional application No. 60/256,370, filed on Dec. 18, 2000.

(51) Int. Cl. A61K 31/66; A61K 9/48; A61K 9/20; A61K 31/255

(52) U.S. Cl. 424/452; 424/465; 514/120; 514/114; 514/517

(57) ABSTRACT

The present invention relates to a male contaceptive composition suitable for oral administration, wherein the composition comprises a pharmaceutically acceptable, non-toxic cationic salt of an alkyl sulfate.

### FIELD OF THE INVENTION

The present invention is directed to a contraceptive composition and a method of using such a composition in human and veterinary medicine for the control of fertility. In particular, the present invention is directed to a male contraceptive composition that is suitable for oral administration.

### BACKGROUND OF THE INVENTION

Contraceptive methods involving the administration of chemical substances are widely practiced among women who desire to limit pregnancies. Such methods control fertility through various biological mechanisms. Among the presently used chemical methods of fertility control, the most important are those which act by means of the following: a) suppression of ovulation through inhibition of gonodotropin release; b) alteration of the female reproductive tract to prevent migration of sperm to the site of fertilization or, if fertilization occurs, to block implantation of the zygote (nidation); or c) spermicidal action.

Oral contraceptives are the most prominent chemical contraceptive agents. These agents are of two types: a) an estrogen combined with a progestin, and b) a progestin alone. The contraceptives of the combined type act primarily by suppressing ovulation by negative feedback to prevent gonadotropin (LH and FSH) release by the hypothalamas, but alterations in the reproductive tract may also contribute to the anti-fertility effect. Such alterations include changes in the cervical mucus (which increases the difficulty of sperm migration) and in the endometrinum (which decrease the likelihood of nidation). The action of a progestin alone in a very low oral dose (the “mini-pill”) appears to involve primarily alterations in the female reproductive tract, but ovulation suppression may also occur. Although oral contraceptives are highly effective, their use is associated with unpleasant side effects (such as nausea, depression, weight-gain, and headache) and an increased long-time risk of severe disease (such as break-through bleeding, spotting, and amenorrhea) are also frequent. A progestin, when administered alone, causes an increased incidence of changes in menstrual patterns, especially a marked increase in the amount and duration of menstrual bleeding.

Besides the oral route of administration, the progestin alone therapy may be administered systemically by various sustained-release dosage forms that include: a) depo-injection (IM) of an insoluble progestin (e.g. medroxyprogesterone acetate), b) a subdermal implant, or c) an intravaginal insert. With these methods of administration, the progestin is absorbed into the body continuously at a very low daily dose, and the systemic effects are similar to those produced by the oral administration of a progestin. However, as with the oral progestins, the sustained release methods may cause serious menstrual flow irregularities.

The intrauterine device (IUD) is the most common alternative to the oral contraceptives. The anti-fertility effect of the IUD is not caused by chemical activity. Instead the material forming the IUD induces a foreign body reaction (irritation) in the contiguous endometrium which appears to interfere in some way with nidation. The use of the IUD is complicated, however, by serious problems including the possibility of intrauterine perforation, pelvic inflammation, discomfort, or aggravated menstrual periods.

The traditional IUD has been modified in an effort to enhance its effectiveness. In one embodiment the IUD has been modified to include metallic copper. The contraceptive action of this device results from the combined effects that copper (which very slowly dissolves in the uterine fluids) has on the blastocyst and on the cervical mucus or endometrium, and the effects of the IUD itself, which causes a foreign body reaction in the endometrium.

Alternatively the IUD can be combined with progesterone to provide a sustained release of progesterone locally within the uterine lumen. In this method the progesterone is incorporated into a chamber within a flexible intrauterine device (IUD) formed from a polymer which is capable of releasing progesterone continuously into the uterine fluids at a slow rate over a prolonged period of time. The progesterone acts primarily locally to produce progestational alterations in the cervical mucus and endometrium. However, the antifertility action may also be caused by the reaction of the endometrium to the device itself (“ID effect”) or by systemic absorption of progesterone through the uterine membrane. Again as with other progestin-only therapies, there is an increased incidence of menstrual flow irregularities. Another disadvantage of this method, is the increased risk of ectopic pregnancy.

Other chemical methods of contraception include the post coital administration of estrogens (e.g. diethylstilbestrol or ethynylestradiol) to prevent nidation or post coital administration of prostaglandins that act as abortifacients. Both of these methods, at present, are limited to emergency situations even though the “morning-after” pill has gained some commercial use , Another group of chemical contraceptive agents are the local spermatocides, such as nonoxynol or octoxynol, which are placed into the vagina immediately prior to coitus in the form of creams, foams, jellies or suppositories. The spermicidal action takes place either in the vagina or elsewhere in the reproductive tract. For the latter to occur, the spermicidal agent is either absorbed in sperm membranes or is transported into the uterus under the influence of uterine contractions. The spermicidal methods are less reliable in preventing pregnancy and are inconvenient to use.

From the foregoing, it is evident that the presently available methods of contraception are inadequate for various reasons because they: a) produce unpleasant side effects or increase risk of serious disease, b) may be unreliable, c) may be inconvenient or intrude on sexual enjoyment, or d) they are all intended for female use only. A need obviously exists for improved methods which combine effectiveness with increase in safety and convenience. A need also exists for a male contraceptive which allows the male to control his own reproduction.

It has been previously reported that a class of alkyl or alkenyl sulfate salts will effectively prevent fertilization when introduced into the uterine lumen or vaginal cavity. See U.S. Pat. Nos. 4,264,575, 4,264,576, 4,264,577, and 4,264,578, the disclosures of which are expressly incorporated herein. Although these compounds were effective when administered directly to the female reproductive tract, oral dosage formulations proved to be ineffective in preventing fertilization. Surprisingly, these compounds have now been found to be effective as male contraceptive agents when the compounds are administered orally or by other means that provide enhanced systemic levels of the compounds in males. In accordance with this discovery, the present invention provides a safe, effective and convenient means for controlling fertility in humans and animals.

### SUMMARY OF THE INVENTION

The present invention is directed to a contraceptive composition comprising a pharmaceutically acceptable cationic salt of a compound having the general formula:

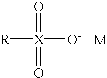

wherein X is P or S;

M is a cation; and °

R is C₄-C₂₄ alkyl, C₄-C₂₄ alkenyl, C₄-C₂₄ alkoxy,

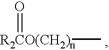

R₃(OCH₂CH₂)m or

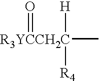

wherein R₂ is C₄-C₂₄ alkyl or C₄-C₂₄ alkoxy, n is 0-4, R₃is C₄-C₂₄alkyl, m is 3-10, R₄ is H or COOR_{6,} R₆ is C₁-C₁₃ alkyl and Y=0 or NH. The invention is also directed to a method of using such a composition for inhibiting conception, and in particular the compositions are formulated for use on male subjects. This will provide the first male oral antifertility agent and opens a new method for family planning.

### BRIEF DESCRIPTION OF THE DRAWINGS

FIG. 1 is a Lineweaver-Burk plot of the inhibition of acrosin by concentrations of tetradecyl sodium sulfate well below the critical micelle concentration of 10⁻⁴ M (Winsor, 1945). BAPNA was used for this analysis and a Ki of 7.82 ×10 M was observed.

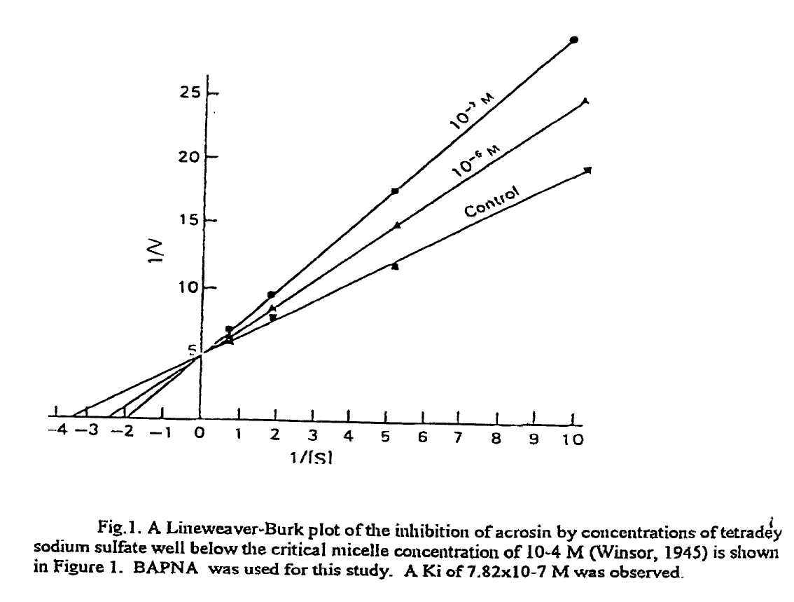

### DETAILED DESCRIPTION OF THE INVENTION

In describing and claiming the invention, the following terminology will be used in accordance with the definitions set forth below.

As used herein, “effective amount” means an amount sufficient to produce a selected effect. For example, an effective amount of a contraceptive agent is an amount of the active agent sufficient to reduce the fertility of an individual.

The general chemical terms used in the description of the compounds of the present invention have their usual meanings. For example, the term “alkyl” by itself or as part of another substituent means a straight or branched aliphatic chain, having the stated number of carbon atoms.

The term “halo” includes bromo, chloro, fluoro, and iodo.

The term, “parenteral” means not through the alimentary canal but by some other route such as subcutaneous, intramuscular, intraspinal, or intravenous.

The present invention is directed to a new method and composition useful in human and veterinary medicine for controlling fertility. Advantageously, the compositions of the present invention block fertilization when they are administered to males, and yet fertility can be restored simply by terminating the treatment. In particular the composition comprises a compound of Formula I:

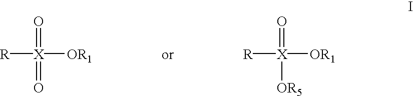

wherein X is P or S;

R is C₄-C₂₄ alkyl, C₄-C₂₄ alkenyl, C₄-C₂₄ alkoxy,

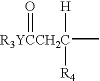

R₃(OCH₂CH₂)ₘO— or 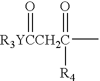 R₁ is H, C₁-C₄ alkyl, a pharmaceutically acceptable cation or

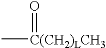

wherein L ranges from 4-24;

R₅ is H, C₁-C₄ alkyl, phenyl or a pharmaceutically acceptable cation,

R₂ is C₄-C₂₄ alkyl or C₄-C₂₄ alkoxy, n is 0- 4;

R₃ is C₄-C₂₄ alkyl, m is 3- 10;

R₄ is H, C₁-C₄ alkyl or COOR₆;

R₆ is C₁-C₁₃ alkyl and Y=O or NH.

This compound is combined with a pharmaceutically acceptable carrier to prepare a contraceptive composition suitable for use in preventing conception in warm blooded vertebrates. The cation used in the composition of the present invention can be any pharmaceutically acceptable, non-toxic cation known to those skilled in the art, including but not limited to sodium, potassium, lithium, calcium, magnesium, copper, aluminum, zinc, pyridinium, substituted pyridinium, ammonium, or substituted ammonium. It will be appreciated by those skilled in the art that when the cation (M) has a valency greater than one, more than one alkyl sulfate moiety will be associated with the cation.

Surprisingly, applicants have discovered that the compounds of Formula I when administered to males in a form that enhances systemic levels of the compounds will inhibit fertility of the males and will function as an effective male contraceptive. Furthermore, in accordance with one embodiment, these compounds are administered orally to males to provide a contraceptive effect without producing hormonal effects either locally or systemically. This is an advantage over oral contraceptives currently used in females. The compounds of the present invention have very low toxicity and the activity last for up to twenty-four hours. Furthermore, the fill restoration of fertility upon ceasing administration of the compounds indicates that the compounds have no permanent effects on the male reproductive system.

The active compounds of the present invention are believed to exert their contraceptive effect by inhibiting (or preventing the activation of) the enzymes necessary for a sperm cell to penetrate the outer membranes of the ovum. In the -male and when deposited in the female, sperm are incapable of fertilizing an ovun since they lack the capacity to penetrate the outer investments. Before fertilization can occur, specific hydrolytic enzymes emanating from the sperm must digest each investment so as to form a passage for sperm penetration. The process by which sperm achieve the ability to penetrate the ovum is known as “capacitation”.

Capacitation involves labilization of sperm membranes and the release, activation, or exposure of the ovum penetrating enzymes as needed to attack each investment. There is evidence that the activation of the ovum penetrating enzymes may involve the removal of specific inhibitors of the enzymes. The exact biochemical transformations occurring during capacitation are not fully understood, but the enzymes must exert their action either while bound to the sperm membranes or upon release from sperm after the sperm and the ovum make contact in the fallopian tube. For a review of the biochemistry of capacitation and of the inhibition of ovum penetrating enzymes see McRorie et.al., Am. Rev. Biochem, 43,777 (1974) and E. S. Hafez, Ed., Human Semen and Fertility Regulation in Men, C. V. Mosby Co., St. Louis, MO., 1976, pg. 201-242 and 563-582.

The compounds of the present invention have activity as inhibitors of acrosin and hyaluronidase, two enzymes that are necessary for penetration of the outer investments of the egg during fertilization. Acrosin is a serine protease found in sperm acrosomes. It is responsible for the dissolution of the zona pellucida layer of the egg. Hyaluronidase, is an acrosomal enzyme responsible for the dissolution of the corona radiata. The compounds represented by Formula I control fertility by binding to sperm membranes and inhibiting, or preventing the activation of, one or more enzymes required during fertilization to allow sperm to penetrate the outer investments of the ovum. An ovum contains three outer investments—the cumulus oophorus, the corona radiata, and the zona pellucida—that are barriers to fertilization. It has been discovered that the alkyl sulfates of Formula I inhibit in vitro the action of acrosin, the sperm acrosomal enzyme which is known to be responsible in vivo for the penetration of the zona pellucida. The compounds of the present invention have also been found to inhibit hyalurinidase which is the enzyme responsible for the penetration of the corona radiate nhibition of acrosin and hyalurinidase in vivo will lead to interruption of the ovum penetration process thereby effectively preventing fertilization and pregnancy. Tetradecyl sodium sulfate salts have been shown to be one of the most effective inhibitors of acrosin and in one preferred embodiment the contraceptive composition comprises a tetradecyl sodium sulfate salt.

Consequently, the compounds of the present invention effectively prevent fertilization by preventing penetration of the egg. Therefore any procedure that enables a sufficient concentration of these inhibitory compounds to be exposed to the sperm acrosomal enzymes can be used as a contraceptive therapy. For example, the compounds can be administered intravaginally as described in U.S. Pat. Nos. 4,264,575, 4,264,576, 4,264,577, and 4,264,578. Although those references report that alkyl sulfonates can be administered to females intravaginally, attempts to formulate an contraceptive oral dosage form failed. Surprisingly, even though the compounds are ineffective as contraceptive agents when administered orally to females, applications have discovered that the compounds are effective contraceptive agents in males when administered orally or in a form that enhances systemic levels of the compounds.

Applications were the first to discover that the compounds of Formula I bind to sperm membranes. Therefore, contact of sperm cells prior to coitus will result in the compounds binding to the sperm and being carried to the right location for binding and inhibiting acrosin and hyaluronidase enzymatic activity when the sperm contact the ovtum. Thus the compounds of the present invention can be administered to males to provide effective contraception using any route of administration that allows for contact of the sperm with an effective amount of the present compounds.

To provide an effective contraceptive effect it is not necessary to prevent the capacitation of all sperm cells. Reducing the number of sperm that are capable of undergoing capacitation to less that three million per milliliter of ejaculate should be sufficient to prevent pregnancy Therefore, in accordance with one embodiment the compounds of the present invention are formulated to be administered to males in a form that provides an effective amount of the active agent to prevent capacitation of a majority of the sperm, and more preferably prevent capacitation in at least 80% to 95% of the individual's sperm cells.

The present invention is directed to pharmaceutical compositions comprsing compounds that inhibit sperm acrosin and hyaluronidase activity. In particular the composition comprises a compound of Formula I.:

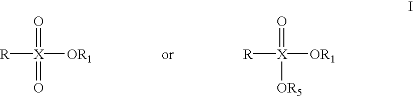

wherein X is P or S; R is C₄-C₂₄ alkyl, C₄-C₂₄ alkenyl, C₄-C₂₄ alkoxy or

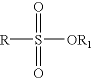

R₁ is H, a pharmaceutically acceptable cation or

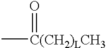

wherein L ranges from 4 to 24;

R₅ is H, C₁-C₄ alky, phenyl or a pharmaceutically acceptable cation; and R₃ is C₄-C₂₄ alkyl.

The cation used in the compositions of the present invention can be any pharmaceutically acceptable, non-toxic cation known to those skilled in the art, including but not limited to sodium, potassium, lithium, calcium, magnesium, copper, aluminum, zinc, pyridinium, substituted pyrdinium, ammonium, or substituted ammonium). It will be appreciated by those skilled in the art that when the cation (M) has a valency greater than one, more than one alkyl sulfate moiety will be associated with the cation.

In accordance with one embodiment the compound has the structure:

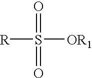

wherein R is C₄-C₂₄ alkyl, C₄-C₂₄ alkoxy or

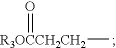

R₁ is H, a pharmaceutically acceptable cation or

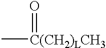

wherein L ranges from 4 to 24; and

R₃ is C₄-C₂₄ alkyl. In one preferred embodiment R is C₁₀-C₁₈ alkyl and R₁ is a pharmaceutically acceptable cation selected from the group consisting of sodium, potassium, lithium and calcium and in one embodiment tho compound is tetradecyl sodium sulfonate. In another preferred embodiment R is C₄-C₂₄ alkyl and R. is

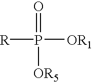

wherein L is 16 to 24, and more preferably L is 20.

Alternatively, in one embodiment, the compound is an sulfoalkyl alkanoate salt of the formula

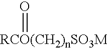

wherein R is a straight chain alkyl group having from 9 to 13 carbon atoms, a branched chain alkyl group having from 9 to 17 carbon atoms, or an alkenyl group having from 9 to 13 carbon atoms, n is 2, 3 or 4 and M is a pharmaceutically acceptable, non-toxic cation.

In another embodiment of the present invention the compound has the structure:

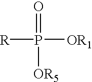

wherein R is C₄-C₂₄ alkyl or C₄-C₂₄ alkoxy;

R₁ is H or a pharmaceutically acceptable cation; and

R₅ is H or C₁-C₄ alkyl. In one preferred embodiment R₁ and R₅ are both H and R is C₄-C₂₄ aLkyl or C₄-C₂₄ alkoxy.

In accordance with one embodiment a composition comprising a compound of Formula I is used to inhibit in vivo the enzymatic activity of sperm acrosin and/or hyaluronidase. The method comprises the steps of contacting the enzyme with a compound of Formula L More particularly, the present invention relates to the use of a composition comprising a compound of Formula I to inhibit the capacitation of sperm cells and thus prevent conception in warm blooded vertebrates. In order to prevent pregnancy, an effective amount of the active compound must be present at the site of fertilization in the fallopian tube when sperm and the ovum make contact prior to penetration of the ovum. Since the compounds of Formula I are known to bind sperm membranes, the sperm cells can be contacted with an effective amount of the compounds before ejaculation and the compounds will be carried with the sperm to the ovum where they will prevent penetration of the ovum outer membranes.

In one embodiment, a compound of Formula I is combined with a pharmaceutically acceptable carrier and administered to a male in a form that produces systemic levels in an amount effective to prevent capacitation of the individual's sperm cells. In this embodiment the active compound binds to the sperm membranes and is carried to the site of action. The contraceptive compositions of the present invention can also be used in combination with any of the known methods of contraception, including the previously described barrier methods and spermicidal creams.

Administration can be effected continuously or intermittently using either a daily oral dosing regiment or by dosing precoitally to provide an amount of the active compound that is effective for its intended purpose. In one preferred embodiment the compositions of the present invention are formulated for oral administration using either a solid or liquid dosage form, and in particular the composition is formulated for use on male subjects. The compounds can be used in combination with one or more conventional pharmaceutical additive or excipients used in the preparation of tablets, capsules, lozenges suspensions, gelcaps and other orally administrable forns. Alternatively, the compositions of The present invention can be administered daily as a suppository or transdermal patch to provide an effective amount of a compound of Fornula I.

Methods of administration are well known to those of skill in the art and include, but are not limited to oral, parenteral or topical administration. More particularly, oral dosage forms of the compounds of Formula I can be administered in the form of tablets, capsules, sugar- or film-coated tablets, liquid solutions or suspensions. In one preferred embodiment the composition is formulated as a gelcap for oral delivery. Dosage forms for parenteral administration, for example intramuscular, intravenous or subcutaneous administration can be formulated in physiological saline using techniques known to those skilled in the art. The composition of the present invention can also be formulated as an ointment, cream, gel, lotions, foams and sprays.

In one embodiment the active agent is administered in a sustained release form that maintains stable systemic levels of an effective amount of a compound of Formula I. For example, the composition can be formulated for delivery by a dermal patch or subdermal implant using standard techniques known to those skilled in the art. It will be apparent to those skilled in the art that transdermal therapy will provide continuous doses of compound for an extended period of time. It will also be understood that these doses can be predicted and regulated. The rate of release from these type devices can be measured in vitro wherein the carrier is placed in water or calcium-free Ringer's solution at 37° C. for successive periods of time (e.g. 24 hrs.), and the amount of active ingredient released after each period is assayed. The same technology could be used for sub-dermal implants or buccal therapy. Those skilled in the art would recognize that any administration which produced sufficient systemic levels to cause binding of the present compounds to the sperm membranes would work to provide an effective contraception effect.

In one preferred embodiment of the present invention the compounds used are alkyl sulfate salts having the formula R-OSO₃ M, wherein R represents an alkyl group and M is a pharmaceutically acceptable, non-toxic cation (e.g.) sodium, potassium, lithium, calcium, magnesium, copper, aluminum, zinc, pyridinium, substituted pyridinium, ammonium, or substituted ammonium). More particularly R is selected from the group consisting of

(a) C₁₁-C₃₀ straight chain alkyl or alkenyl;

(b) C₁₀-C₃₀ branched chain alkyl or alkenyl, the alpha carbon of which is not branched; or

(c) C₁₃-C₃₀ branched chain alkyl or alkenyl, the alpha carbon of which is branched. More particularly, one preferred compound is tetradecyl sodium sulfonate.

It will be appreciated by those skilled in the art that when the cation (M) has a valency greater than one, more than one alkyl sulfate moiety will be associated with the cation. The alkyl sulfates are known in the art. It will also be appreciated by those skilled in the art that while alkyl sulfates are one preferred group of compounds suitable for use with the present invention other compounds represented by Formula I, including alkyl sulfonates, phosphates, phosphonates, sulfopropyl esters and others will also act as acrosin inhibitors in the same way as the alkyl sulfates.

The pyridinium sulfate salts of the alkyls are prepared by reacting the desired alkyl with pyridine sulfate trioxide according to the method of A. Sobel et al., J. Am. Chem. Soc. 63, 1259 (1941). Other salts can be prepared by methods well known in the art of chemistry. The alkyl sodium sulfates were prepared by sulphation of >98% pure alcohol of C₄-C₂₄ using chlorosulphonic acid according to the procedure of Dreger, Keim, Miles, Shedlovsky and Ross (1944). An example was sulphation of 1- Tetradecanol purchased from Continental Oil Co. using chlorosulphonic acid produced material that was 99.6% pure by gas chromatography. The alkyl sulfates can be assayed according to the procedure of Hagashi, Anal. Biochem., 67, 503 (1975).

Additional compounds similar in structure to the alkyl sulfates have been shown to have activity as acrosin and hyaluronidase inhibitors and therefore are anticipated to have activity in male contraceptive formulations. For example, compounds of the formula: R₇(OCH₂CH₂)ₙ—OH, 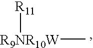 and ArSO₃M

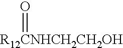

wherein R₇ is C₆-C₃₀ alkyl and n ranges from 5-10;

R₉ is C₁₀-C₂₄ alkyl, R₁₀ and R₁₁ are independently H or C₁-C₄ alkyl, and W is a pharmaceutically acceptable anion; R₁₂ is C₈-C₃₀ alkyl; Ar is an aromatic moiety such as phenyl, alkyl phenyl, naphthyl, 2 or 3-benzofuranyl, and M is a pharmaceutically acceptable cation, are anticipated to have activity as male contraceptive agents.

The ability of the compounds of Formula I and structurally similar compounds, to prevent digestion of the zona pellucida is demonstrated by an in vitro test. The test comprises observing an isolated rabbit ovum under a microscope while incubating the ovum in calcium free Ringer's solution in the presence of acrosin, with and without the alkyl sulfate being present in the medium. In the absence of alkyl sulfate, complete removal of the zona pellucida is observed. With compound present, the zona pellucida remains substantially intact.

In addition, the antifertility effects of the compounds of Formula I have been measured by standard test procedures whereby the test compound is introduced orally in male rabbits (see Example 4). The animals (of proven fertility) are then bred and the genital system of the females is examined to determine the number of embryos. Animal experiments have shown that tetradecyl sodium sulfonate has an LD₅₀ of 140 mg/kg. This relates to about 10 grams in a 150-pound person. The daily dose used in these experiments has been the equivalent of approximately 100 mg for a 150-pound person. This is 100 times less than the LD50 for this compound. Accordingly, the present compounds and its derivatives should be an effective oral contraceptive for males.

### EXAMPLE 1

Acrosin Inhibition In Vitro

The inhibition of acrosin is assessed in vitro using benzoyl arginine p-nitroanilide (BAPNA) as the enzyme substrate according to the following procedure. Acrosin (200 μl, purified from boar sperm) and a solution of the test compound (200 μl, 10 mg/ml., or 50 μl, 1 mg/ml) in 0.05 M triethanolamine buffer, pH 7.8, are incubated at room temperature for 5 minutes. A control using the buffer solution without the test compound added is also run. A 200 μl-sample of the incubation mixture is then withdrawn and is added to a cuvettecontaining BAPNA (1 ml) and triethanolamine buffer (2 ml). The mixture is stirred and placed in a Gilford recording spectrophotometer. The increase in optical density at 383 mn is measured. One unit of acrosin activity is defined as the amount of acrosin which will cause an increase in optical density at 383 mn of 0.001/minute. One unit of inhibitory activity is defined as the amount of inhibitor which will cause a reduction in the increase in optical density at 383 nm of 0.001/minute.

The results of the testing of sodium alkyl sulfates for acrosin inhibition using the above described procedure are shown below in Table I.

TABLE I
In vitro acrosin inhibition of Na alkyl or alkenyl sulfates.
A. Sodium Salts: RSO₃Na

| Compound | Conc. of | |
|---|---|---|
| R of | Test Solution | Inhibition |
| Formula I | (Mg/ml) | (units/mg.) |
| n-hendecyl | 10 | 0 |
| n-decyl | 10 | 0 |
| n-tridecyl | 10 | 3,200 |
| n-tetradecyl | 10 | 14,200 |
| ″ | 1 | 960,000 |
| n-pentadecyl | 10 | 14,300 |
| n-hexdecyl | 10 | 15,200 |
| n-heptadecyl | 10 | 18,300 |
| n-octadecyl | 10 | 26,000 |
| n-eicosyl | 10 | 31,800 |
| n-docosyl | 1 | 300,000 |
| n-tetracosyl | 1 | 123,000 |
| n-heptacosyl | 1 | 100,000 |
| 2-tetradecyl | 1 | 87,000 |
| 4-tetradecyl* | 1 | 102,000 |
| 6-tetradecyl | 1 | 90,000 |
| 7-tetradecyl | 1 | 112,000 |
| 7-hexadecyl | 1 | 123,000 |
| 8-hexadecyl | 1 | 28,000 |
| 9-octadecyl | 1 | 148,000 |
| 2-octyldodecyl | 1 | 132,000 |
| 3,7,11-trimethyldodecyl | 1 | 29,000 |
| tetrahydrogeranyl | 1 | 240,000 |
| 12-methyltridecyl | 1 | 172,000 |
| myristoleyl | 1 | 160,000 |
| myristeladyl | 1 | 216,000 |
| oleyl | 1 | 152,000 |
| linoleyl | 1 | 210,000 |
| 12-methyltridecane-9-yl | 1 | 152,000 |

B. Other salts: ROSO₃M

| Compound | | | Inhibition |
|---|---|---|---|
| R of | M of | Conc. of | (units/mg) |
| Formula I | Formula I | Test Sol. | (mg/ul) |
| CH₃(CH₂)₁₃— | Ca | 10 | 48,000 |
| — | K | 10 | 62,000 |
| — | Mg | 10 | 59,000 |
| — | Li | 10 | 58,000 |
| — | NH₄ | 10 | 47,000 |
| — | pyridinium | 10 | 48,000 |
| CH₃(CH₂)₁₁— | diethanol- | 10 | 90,000 |
| | ammonium | | |
| — | triethanol- | 10 | 90,000 |
| | ammonium | | |

C. Phosphoric acid derivatives:

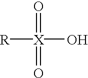

| Compound | Conc. of | |
|---|---|---|
| R of | Test Solution | Inhibition |
| Formula I | (Mg/ml) | (units/mg.) |
| C₁₀H₂₁ | 1 | 130,000 |
| C₁₂H₂₅ | 1 | 120,000 |
| C₁₄H₂₉ | 1 | 114,000 |
| C₁₈H₃₇ | 1 | 360,000 |

*Tested as potassium salt.

### EXAMPLE 2

Hyaluronidase Inhibition In Vitro

The inhibition of hyaluronidase was assessed using chondroitin sulfate as the enzyme substrate according to the following procedure. Agar plates containing chondroitin sulfate (0.1 percent) were prepared by the following method: water solutions of I percent agarose, 2 M sodium chloride (0.1 vol.), 1 M sodium acetate (pH 5.0, 0.1 vol), and sodium azide (0.5 mg/ml, 0.1 vol.) qe mixed and the mixture is brought to the boiling temperature. After cooling to about 65° C., a solution of chondroitin sulfate (10 mgtml, 0.1 vol.) is added. After cooling to about 45° C., the resulting mixture (20 ml) is poured onto a petri dish. The plates are allowed to cool to room temperature. A well 2 mm in diameter is then cut in the agar surface. Testicular bovine hyaluronidase (Sigma Chemical Company) (5 [I, 10 mgcml or 1 mg/ml) and a solution of the test compound (5 ttl, 10 mg/ml) in I M sodium acetate buffer, pH 5.0, are added to the well. A control using only the buffer solution without added test compound is also run. The plates are then incubated overnight (about 16 hours) at 37° C. The zones of hydrolysis of chondroitin sulfate by hyaluronidase are visualized by flooding the plate with 10 percent cetyl trimethyl ammonium bromide. The area of the zone is logarithmically proportional to the concentration of hyaluronidase. Inhibitor activity is determined by comparing the area of hydrolysis from the test compound to the area of hydrolysis from the control. The results are expressed as percent inhibition calculated as follows:

$$percent inhibition = 100 - \frac{\begin{matrix}area of zone of inhibition \\ for test compound\end{matrix}}{\begin{matrix}area of zone of inhibition \\ for test control\end{matrix}}$$

When tested according to the above described method the sodium alkyl sulfates of Formula I gave the following results:

TABLE II
In vitro hyaluronidase inhibition of
alkyl or alkenyl sulfate salts
A. Sodium Salts: RSO₃Na

| Compound | Conc. of | |
|---|---|---|
| R of | Test Solution | % |
| Formula I | (Mg/ml) | Inhibition |
| n-hendecyl | 10 | 100 |
| n-dodecyl | 10 | 71 |
| n-tridecyl | 10 | 80 |
| n-tetradecyl | 10 | 82 |
| n-pentadecyl | 10 | 60 |
| n-hexadecyl | 10 | 75 |
| n-heptadecyl | 10 | 23 |
| n-octadecyl | 10 | 30 |
| n-eicosyl | 10 | 0 |
| n-docosyl | 1 | 0 |
| n-tetracosyl | 1 | 0 |
| n-heptacosyl | 1 | 0 |
| 2-tetradecyl | 1 | 100 |
| 4-tetradecyl | 1 | 0 |
| 6-tetradecyl | 1 | 12 |
| 7-tetradecyl | 1 | 0 |
| 7-hexadecyl | 1 | 0 |
| 8-hexadecyl | 1 | 0 |
| 9-octadecyl | 1 | 7 |
| 2-octyldodecyl | 1 | 0 |
| 3,7,11-trimethyldodecyl | 1 | 39 |
| tetrahydrogeranyl | 1 | 0 |
| 12-methyltridecyl | 1 | 7 |
| myristoleyl | 1 | 13 |
| myristeladyl | 1 | 13 |
| oelyl | 1 | 0 |
| linoleyl | 1 | 0 |
| 12-methyltrideccen-9-yl | 1 | 38 |

B. Other salts: ROSO₃M

| | |
|---|---|
| Compound | Conc. of |

| R of | M of | Test Sol. | % |
|---|---|---|---|
| Formula I | Formula I | (mg/ul) | Inhibition |
| CH₃(CH₂)₁₃— | Ca | 10 | 38 |
| | K | 10 | 27 |
| | Mg | 10 | 14 |
| | Li | 10 | 27 |
| | NH₄ | 10 | 14 |
| | pyridinium | 10 | 14 |
| CH₃(CH₂)₁₁— | diethanol- | 10 | 100 |
| | ammonium | | |
| | triethanol- | 10 | 100 |
| | ammonium | | |

### EXAMPLE 3

The following compounds have also been found to exhibit acrosin or hyaluronidase activity as indicated. A denotes acrosin inhibition and H denotes hyaluronidase inhibition. Furthermore, compound nos. 6, 20 and 31 have shown activity as male contraceptives when administered to male rabbits in accordance with the procedures of Example 4.

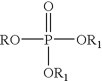

| | |
|---|---|
| Compound | Structure |

| No. | R | R₁ | Activity |
|---|---|---|---|
| 1 | C₁₀H₂₁ | Ph | A |
| 2 | C₁₄H₂₉ | Ph | A |
| 3 | C₁₈H₃₇ | Ph | A |
| 4 | 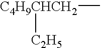 | Ph | H |
| 5 | C₁₀H₂₁ | H | A (1.3 × 10⁵ u/mg) |
| 6 | C₁₂H₂₅ | H | A (1.2 × 10⁵ n/mg) |
| 7 | C₁₈H₃₇ | H | A (3.6 × 10⁵ u/mg) |
| 8 | C₁₄H₂₉ | H | A (1.1 × 10⁵ u/mg) |

R—(OCH₂CH₂)ₙOSO₃Na

| | |
|---|---|
| Compound | Structure |

| No. | R | n | Activity |
|---|---|---|---|
| 9 | C₁₂H₂₉ | 1-4 (average) | A, H |

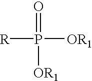

| | |
|---|---|
| Compound | Structure |

| No. | R | R₁ | Activity |
|---|---|---|---|
| 10 | C₆H₁₃ | H | A, H |
| 11 | C₈H₁₇ | H | A, H |
| 12 | C₁₀H₂₁ | H | A |
| 13 | C₁₂H₂₅ | H | A |
| 14 | C₁₄H₂₉ | H | H |
| 15 | C₁₂H₂₅C₆H₄CH═CH— | Na | A, H |

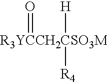

| Compound | Structure | | | | |
|---|---|---|---|---|---|
| No. | R₃ | Y | M | R₄ | Activity |
| 16 | C₆H₁₃ | O | Na | COOC₆H₁₃ | A |
| 17 | C₁₃H₂₇ | O | Na | COOC₁₃H₂₇ | A |
| 18 | C₂₂H₄₅ | O | Na | H | A |
| 19 | C₁₄H₂₉ | O | Na | H | A |
| 20 | C₁₈H₃₇ | O | Na | H | A |
| 21 | C₁₂H₂₅ | NH | Na | CH₃ | A |

R₁₅-(OCH₂CH₂)ₙOH

| | |
|---|---|
| Compound | Structure |

| No. | R₁₅ | n | Activity |
|---|---|---|---|
| 22 | olely | 5 (average) | A |
| 23 | olely | 10 (average) | A |

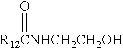

| Compound | Structure | |
|---|---|---|
| No. | R₁₂ | Activity |
| 24 | C₁₁H₂₃ | |

R₉N(R₁₁)₃⁺W^{- and (R}₉)₂N(R₁₁)₂⁺W⁻

| Compound | Structure | | | |
|---|---|---|---|---|
| No. | R₉ | R₁₁ | W | Activity |
| 25 | C₁₄H₂₉ | CH₃ | Br | H |
| 26 | C₁₈H₃₇ | CH₃ | Cl | H |
| 27 | C₁₈H₃₇ | C₄H₉ | C₁₈H₃₇OSO₃ | A, H |

The following compounds have also been found to have acrosin inhibition
activity:

| | |
|---|---|
| | Cmp 28 |
| 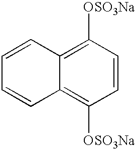 | |
| | Cmp 29 |
| 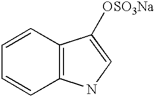 | |
| | Cmp 30 |
| 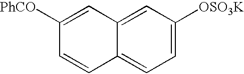 | |

as well as

Compound 31: a ethyl a sulfodocosanoate;

Compound 32: sulfoisopropyl oleate Na salt; and

Compound 33: sulfoglycerol trioleate Na salt.

### EXAMPLE 4

Antifertility Effects In Vivo

Dutch Belted male rabbits of proven fertility were randomly selected. They were given 10 mg/kg tetradecyl sodium sulfate suspended in Ca++ free Ringer's solution by gavage. Control animals were given Ca++ free Ringer's solution by gavage. The male rabbits were dosed each day for 3 days and they were bred to females 2 hours after dosing and again 8 hours after dosing. The female rabbits were then given 100 IU human chorionic gonadotrophin (HCG) to stimulate ovulation. Twenty four hours after the last dose of tetradecyl sodium sulfate the male rabbits were bred and the females treated as before. After twelve days the female rabbits were sacrificed and examined for embryos. The results are summarized in Table III

TABLE III
Inhibition of fertilization by Tetradecyl Sodium
Sulfate (TDSS) Orally in Males

| Treatment | Number Animals | Number Embryos | Embryos (±1.7) |
|---|---|---|---|
| Control | 6 | 34 | 5.7 ± 1.7 |
| TDSS-2 hr. | | | |
| Day 1 | 6 | 0 | 0 |
| Day 2 | 6 | 4 | 0.7 ± 0.5 |
| Day 3 | 6 | 0 | 0 |
| TDSS-8 hr. | | | |
| Day 1 | 6 | 0 | 0 |
| Day 2 | 6 | 3 | 0.5 ± 0.3 |
| Day 3 | 6 | 0 | 0 |
| TDSS-24 hr. | 6 | 31 | 5.2 ± 2.2 |

### EXAMPLE 5

Binding of Tetradecyl Sodium Sulfate Binding of tetradecyl sodium sulfate was assessed by incubating washed sperm with tetradecyl sodium sulfate at 37° C. The amount of tetradecyl sodium sulfate bound to sperm was measured using methylene blue according to the method of Hagashi (1975). Binding was confirmed using tritium-labeled tetradecyl sodium sulfate. The sperm were incubated with [H ]³—tetradecyl sodium sulfate for 10 minutes and then washed 3 times in Ca++ free Ringer's solution. The washed sperm were then resuspended in Ca++free Ringer's solution and layered onto an agarose plate prepared with 1.5 % agarose. After standing for 30 minutes the plates were overlaid with LKB ultrafilm and incubated in a dark room for 2 weeks. The film was developed using standard procedures and examined. Tetradecyl sodium was observed to bind to the entire sperm plasma membranes and not just to acrosomes.

1. An oral contraceptive composition comprnsing a compound of the formula: 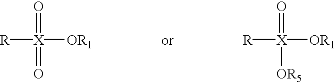
wherein X is F or S;
R is C₄-C₂₄ alkyl, C₄-C₂₄ alkenyl, C₄-C₂₄ alkoxy, 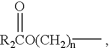
R₃(OCH₂CH₂)ₘO— or 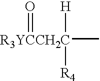
R₁ is H, C₁-C₄ alkyl, a pharmaceutically acceptable cation or 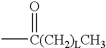
wherein L is 4 to 4 ;
R₅ is H, C,-C₄ alkyl, phenyl or a pharmaceutically acceptable cation, R₂ is C₄-C2₄ allyl or C₄-C2₄ alkoxy, n is 0 to 4;
R₃ is C₄-C₂₄ alkyl, m is 3 to 10;
R4 is H, C,-C₄ alkyl or COOR6; R6 is H or C,-C,₃ alkyl; and
Y=0 or nh and a pharmaceutically acceptable carrier suitable for oral delivery:

2. The composition of claim 1 wherein the composition is formulated as tablet or capsule.

3. The composition of claim 1 wherein the composition is formulated as a gelcap or suspension.

4. The composition of claim 1 wherein the compound has the general formula 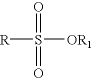
wherein R is C₄-C₂₄ alkyl, C₄-C₂₄ alkoxy, or 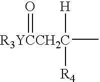
and Y is O.

5. The composition of claim 4 wherein
R is C₁₀-C₁₈ alkyl;
R₁ is a pharmaceutically acceptable cation or 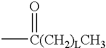
and L is 20.

6. The composition of claim 4 wherein R is 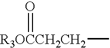
R₃ is C₄-C₂₄ alkyl; and
R₁ is a pharmaceutically acceptable cation.

7. The composition of claim 1 wherein the compound has the general formula 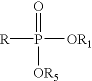
wherein R is C₄-C₂₄ alkyl or C₄-C₂₄ alkoxy; and
R₁ and R5 are independently H or a pharmaceutically acceptable cation.

8. The composition of claim 7 wherein
R is C₄-C₂₄ alkoxy;
R₁ is H or a pharmaceutically acceptable cation; and
R₅is H.

9. The contraceptive composition of claim 1 wherein the compound has the general formula: 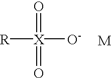
wherein X is P or S;
M is a cation; and
R is C₄-C₂₄ alkyl, C₄-C₂₄ alkenyl, C₄-C₂₄ alkoxy, 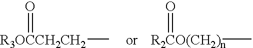
wherein n=2, 3 or 4;
R₂ =C₉-C,₃ alkyl; and
R₃ =C₄-C₂₄ aLkyl.

10. The composition of claim 9 wherein X is S.

11. The composition of claim 9 wherein the composition is formulated as tablet or capsule.

12. The composition of claim 9 wherein the composition is formulated as a Gelcap or suspension.

13. The composition of claim 10 wherein the cation is sodium.

14. A contraceptive trnnsdennal patch or implat, wherein said patch or implant comprises a composition comprising a compound of the general formula: 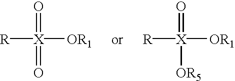
wherein X is P or S;
R₅ is C₄-C₂₄ alkyl, C₄-C₂₄ alkenyl, C₄-C₂₄ alkoxy, 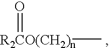
R₃(OCH₂CH₂)ₘO— or 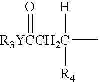
R₁ is H, C₁-C₄ alkyl, a pharmaceutically acceptable cation 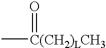
wherein L is 4 to 24;
R₅ is H, C₁-C₄ alkyl, phenyl or a pharmaceutically acceptable cation,
R₂ is C₄-C₂₄ alkyl or C₄-C₂₄ alkoxy, n is 0 to 4;
R₃ is C₄-C₂₄ alkyl, m is 3 to 10;
R₄ is H₁- C₄ alkyl or COOR_{6;}
R₆ is C₁-C₁₃ alkyl; and
Y=O or NH.

14. A method of inhibiting the capacitation of sperm cells, said method comprising the steps of contacting the sperm cells with a composition comprising a compound of the general formula: 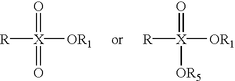
wherein X is P or S;
R is C₄-C₂₄ alkyl, C₄-C₂₄ alkenyl, C₄-C₂₄ alkoxy, 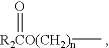
R₃(OCH₂CH₂)ₘO— or 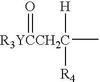
R₁ is H, C₁-C₄ alkyl, a pharmaceutically acceptable cation or 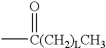
wherein L is 4 to 24;
R is H, C₁-C₄ alkyl, phenyl or a pharmaceutically acceptable cation,
R₂ is C₄-C2₄ alkyl or C₄-C₄ alkoxy, n is 0 to 4;
R₃ is C₄-C2₄ allyl, m is 3 to 10;
R₄ is H, C₁-C₄ alkyl or COOR6;
R is C,-C,₃ alkyl; and
Y =0 or NH and a pharmaceutically acceptable carrier.

15. The method of claim 14 wherein the step of contacting the sperm cells with the compound occurs in vivo in the male.

16. A method for irubiting conception, comprising
administering to a male an effective amount of a pharmaceutically acceptable cationic salt of a compound of the general formula: 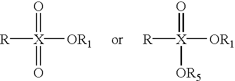
wherein X is P or S;
R is C₄-C₂₄ alkyl, C₄-C₂₄ alkenyl, C₄-C₂₄ alkoxy, 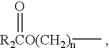
R₃(OCH₂CH₂)ₘO— or 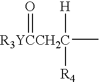
R₁ is H, C₁-C₄ alkyl, a pharmaceutically acceptable cation or 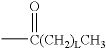
wherein L is 4 to 24;
R₅ is H, C₁-C₄ alkyl, phenyl or a pharmaceutically acceptable cation,
R₂ is C₄-C₂₄ alkyl or C₄-C₂₄ alkoxy, n is 0 to 4;
R₃ is C₄-C₂₄ alkyl, m is 3 to 10;
R₄ is H, C₁-C₄ alkyl or COOR₆;
R₆ is C₁-C₁₃ alkyl;
Y=O or NH.

17. The method of claim 16 wherein the composition is administered orally in a daily dosage form.

18. The method of claim 16 wherein the composition is administered trsdermally or subcutaneously to maintain constant systemic levels for an extended time period.
